(19) United States
(12) Patent Application Publication
(10) Pub. No.: US 2010/0239690 A1
(43) Pub. Date: Sep. 23, 2010

(54) COMPOSITION FOR ORAL CAVITY AND SKIN

(76) Inventor: Satoshi Noda, Kanagawa (JP)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 12/733,731

(22) PCT Filed: Sep. 19, 2008
(86) PCT No.: PCT/JP2008/002596

§ 371 Date: Mar. 17, 2010

(30) Foreign Application Priority Data
Sep. 21, 2007 (JP) 2007 245657

(51) Int. Cl. A61K 33/08; A61K 31/5383; A61K 31/4375; A61K 31/497; A61K 31/4164; A61K 31/155; A61K 31/405; A61K 31/196; A61K 31/573; A61P 29/00; A61P 31/04

(52) U.S. Cl. 424/693; 514/230.2; 514/300; 514/253.08; 514/398; 514/634; 514/420; 514/567; 514/171

(57) ABSTRACT

A composition for oral cavity and skin which contains an antibacterial agent and does not have adverse side effects due to steroids, is provided.

The composition contains 0.01 to 4.5% by mass of at least one selected from the group consisting of an antibacterial agent, an antiviral agent, an anti-HIV agent, a non-nucleic acid-based reverse transcriptase inhibitor, an anticancer agent for external use and a disinfectant; 0.01 to 4.5% by mass of a non-steroidal anti-inflammatory agent; 0.001 to 4.5% by mass of a steroidal anti-inflammatory agent; and 0.001 to 10% by mass of a highly water-absorbent polymer or a cellulose derivative.

### TECHNICAL FIELD

The present invention relates to a composition for oral cavity and skin, and more particularly, to a composition for skin and oral cavity containing at least one or more antibacterial agents and the like, which is formed by incorporating a non-steroidal anti-inflammatory agent in addition to a steroidal anti-inflammatory agent, and has an excellent therapeutic effect for periodontal diseases and dermatitis, while being free of adverse side effects.

### BACKGROUND ART

Steroids strongly suppress inflammation by suppressing the function of lymphocytes or through the action of suppressing inflammatory peptides or suppressing fibroblast proliferation, and are therefore widely used for the treatment of inflammatory diseases. Thus, steroids are known as the most potent anti-inflammatory agents against intraoral diseases such as periodontitis and stomatitis, or dermatitis. Particularly, corticosteroids are anti-inflammatory drugs that are most widely used in both acute inflammation and chronic inflammation.

However, steroids are known to have various adverse side effects. For example, when used in the oral cavity, steroids have adverse side effects such as pain at the site of administration, mucosal disorder, mucosal paresthesia and hypersensitivity. Furthermore, steroid dermatitis in which the epidermis is thinned, thereby readily bleeding or becoming vulnerable to stimulation, and weakening of the resistance to infection because of the suppression of the function of lymphocytes, as well as immunosuppression, digestive tract ulcers and the like are also listed as the adverse side effects of steroids. Systemic edema and urinary retention due to suppression of urinary discretion of sodium, fracture due to acceleration of the elution of bone calcium, and the like are also included in the adverse side effects of steroids. The steroids used for the treatment of inflammatory diseases are medicines prepared by incorporating steroid hormones, and in many cases, glucocorticoids are used. However, the adverse side effects of these glucocorticoids occur at doses that are significantly lower than the doses required for the anti-inflammatory action.

However, antiphlogistic analgesic preparations for external use containing a non-steroidal antiphlogistic analgesic agent and/or a steroidal antiphlogistic analgesic agent as medicinal components, and a base component, have been traditionally used (Patent Document 1).

In recent years, there is available a toothpaste containing medicinal components, and for example, the toothpaste contains an antibacterial agent for the prevention of periodontitis and an abrasive for obtaining a teeth whitening effect. However, due to the characteristic of containing an abrasive, brushing the teeth for a long time causes excessive abrasion of the teeth, and thus, there is a problem that the enamel is peeled off, and conversely, the teeth are brought to the state of being susceptible to dental caries. Furthermore, the inventors of the present invention found that if the pH in the oral cavity is acidic, enamel becomes prone to dissolve, causing the teeth to be susceptible to dental caries (the pH at which enamel starts to dissolve is about 5.5). However, since an absolutely large number of beverages (pH 5 to 7) and foods have acidic pH, there is a problem that the oral cavity is in a circumstance that is easily inclined to become acidic, and dental caries occurs despite brushing of teeth.

Patent Document 1: Japanese Patent Application Laid-Open (JP-A) No. 05-246892

### DISCLOSURE OF INVENTION

### Problem to be Solved by the Invention

The present invention was made as a result of paying attention to such problems of the related art as described above, and is a composition for skin and oral cavity, containing at least one or more antibacterial agent and the like. It is an object of the present invention to reduce adverse side effects of steroids as far as possible, by incorporating a non-steroidal anti-inflammatory agent in addition to a steroidal anti-inflammatory agent, and when the composition is used in the oral cavity, the pH in the oral cavity is maintained to be alkaline (pH being greater than 7.0 to 14) to prevent dental caries, by adding calcium hydroxide, calcium oxide or a calcium compound which is prone to release calcium ions, which compounds are prone to turn into alkali ions, at a proportion of 0.001 to 1.0% by mass. It is also intended that particularly when the composition of the present invention is used in toothpaste, a highly water-absorbent polymer which is soft and thus does not cause wear, is used instead of a generally used abrasive such as silicic acid, calcium carbonate or calcium phosphate (teeth are worn away) so as to prevent excessive abrasion, so that dental plaque attached to the teeth is surface-activated, and the polymer particles enhance the cleaning effect. Furthermore, it is another object to induce a water-supplying action at an affected area through the synergistic action of the medicinal component and the highly water-absorbent polymer or a cellulose derivative, so as to cause discharge of body fluid at the inflamed area and to further accelerate healing of the affected area.

### Means for Solving Problem

A first embodiment of the composition of the present invention is directed to a composition for oral cavity and skin, containing 0.01 to 4.5% by mass of at least one selected from the group consisting of an antibacterial agent, an antiviral agent, an anti-HIV agent, a non-nucleic acid-based reverse transcriptase inhibitor, an anticancer agent for external use and a disinfectant; 0.01 to 4.5% by mass of a non-steroidal anti-inflammatory agent; 0.001 to 4.5% by mass of a steroidal anti-inflammatory agent; and 0.001 to 10% by mass of a highly water-absorbent polymer or a cellulose derivative.

A second embodiment of the composition of the present invention is directed to the composition for oral cavity and skin according to claim 1, which contains 0.001 to 10% by mass of a gelling agent and 0.001 to 1.0% by mass of calcium hydroxide, calcium oxide or a calcium compound that is prone to release calcium ions, in substitution of the highly water-absorbent polymer.

A third embodiment of the composition of the present invention is directed to the invention according to claim 1, wherein the above antibacterial agent was chosen among aspoxicillin, amoxicillin, ampicillin, ciclacillin, talampicillin, sultamicillin tosilate, bacampicillin, pivmecillinam, piperacillin sodium, phenethicillin potassium, benzylpenicillin potassium, benzyl-penicillin benzathine, lenampicillin, cefaclor, cefazolin sodium, cefatrizine propylene glycol, cefadroxil, cefalexin, cefalothin sodium, cefixime, cefepime, cefodizime sodium, cefotaxime sodium, cefotiam, cefotiam hexetil, cefotetan, cefozopran, cefoperazone sodium, cefcapene pivoxil, cefditoren pivoxil, cefdinir, cefsulodin sodium, ceftazidime, ceftizoxime sodium, ceftibuten, cefteram pivoxil, ceftriaxone sodium, cefpiramide sodium, cefpirome, cefbuperazone sodium, cefpodoxime proxetil, cefminox sodium, cefmetazole sodium, cefmenoxime, cefroxadine, cefuroxime axetil, flomoxef sodium, latamoxef sodium, aztreonam, carumonam sodium, imipenem.cilastatin sodium, panipenem.betamipron, biapenem, meropenem, doripenem, faropenem sodium, astromicin, amikacin, arbekacin, isepamicin, kanamycin, gentamicin, erythromycin, sisomicin, dibekacin, streptomycin, spectinomycin, vancomycin, nalidixic acid, nafcillin, oxacillin, levofloxacin, tosufloxacin, gatifloxacin, sparfloxacin, Garenoxacin, enoxacin, ciprofloxacin, ofloxacin, cinoxacin, sulfamethoxazole.trimethoprim, norfloxacin, pazufloxacin, pipemidic acid, pipemidic acid trihydrate, fleroxacin, prulifloxacin, linezolid, lomefloxacin, telithromycin, sulfadimethoxine, sulfamonomethoxine, alumino-p-aminosalicylate, isoniazid, ethionamide, ethambutol, enviomycin, cycloserine, calcium para-aminosalicylate, pyrazinamide, rifampicin, clofazimine, diaphenylsulfone, pentamidine isetionate, amphotericin B, nystatin, flucytosine, miconazole, itraconazole, fluconazole, fosfluconazole, voriconazole, terbinafine, micafungin sodium, griseofulvin, metronidazole and tinidazole group; is at least one,

the above antiviral agent is aciclovir, valaciclovir, vidarabine, ganciclovir, valganciclovir hydrochloride, foscarnet sodium hydrate and palivizumab group; is at least one; and above antiHIV agent was chosen among the zidovudine, didanosine, zalcitabine, sanilvudine, lamivudine, abacavir, tenofovir disoproxil fumarate, disoproxil and emtricitabine group at least one, and above non-nucleoside reverse transcriptase inhibitor is efavirenz, nevirapine, protease inhibitor or saquinavir mesilate, saquinavir, ritonavir, praziquantel and sodium antimonyl tartrate, and above anticancer agent was chosen among fluorouracil, tegafur, bleomycin and the BCG (bacillus calmette and Guerin) group that it is at least one, and an above sterilizer was chosen among Acrinol, the fourth grade ammonium salt, cetylpyridinium chloride, benzalkonium chloride, sodium hypochlorite, calcium hypochlorite, chlorhexidine, Irgasan, phenol and the iodine group that it is at least one.

A fourth embodiment of the composition of the present invention is directed to the invention according to claim 1, wherein the non-steroidal anti-inflammatory agent was chosen among indomethacin, diclofenac, acemetacin, alclofenac, amfenac, aspirin, bendazac, benorylate, benoxaprofen, bucloxic acid, bufexamac, bumadizone, butibufen, carprofen, cinmetacin, clidanac, clometacin, cloripac, diclofenac, diflunisal, etodolac, etofenamate, felbinac, fenbufen, fenclofenac, fenclorac, fendosal, fenoprofen, fentiazac, flufenamic acid, flurbiprofen, glafenine, ibufenac, ibuprofen, indomethacin, isofezolac, isoxepac, ketoprofen, ketorolac, lonazolac, meclofenamic acid, mefanamic acid, metiazinic acid, nabumetone, naproxen, niflumic acid, oxametacin, oxaprozin, pirazolac, piroxicam, protizinic acid, salicylic acid, sulindac, surgam, tenidap, tenoxicam, tiaramide, tinoridine, tolfenamic acid and tolmetin group that it is at least one.

A fifth embodiment of the composition of the present invention is directed to the invention according to claim 1, wherein the steroidal anti-inflammatory agent was chosen among dexamethasone, betamethasone, cortisone, hydrocortisone, fludrocortisones, methylprednisolone, triamcinolone, paramethasone and prednisone group that it is at least one.

A sixth embodiment of the composition of the present invention is directed to the invention according to claim 1, wherein the highly water-absorbent polymer was chosen among carboxyvinyl polymers, polyvinyl alcohol, polyacrylate, starch-acrylic acid graft, isobutylene-maleic acid and poly-N-vinylacetamide group that it is at least one.

A seventh embodiment of the composition of the present invention is directed to the invention according to claim 1, which further contains 0.001 to 1.0% by mass of calcium ions, and has a pH of greater than 7 to 14.

An eighth embodiment of the composition of the present invention is directed to the invention according to claim 1, which further contains 0.001 to 20% by mass of at least one antihistamine agent selected from the group consisting of chlorpheniramine maleate, diphenhydra

mine hydrochloride, clemastin fumarate, triprolidine hydrochloride, alimemazine tartrate, promethazine hydrochloride, homochlorcyclizine hydrochloride, hydroxyzine and cyproheptadine hydrochloride.

A ninth embodiment of the composition of the present invention is directed to the composition according to claim 1, which further contains 0.01 to 20% by mass of at least one antiphlogistic agent selected from the group consisting of tacrolimus hydrate, ethyl aminobenzoate, bismuth subgallate, camphor, zinc chloride and crotamiton.

### EFFECT OF THE INVENTION

According to the first and second embodiments of the present invention, when a non-steroidal anti-inflammatory agent is used together with an antibacterial agent, an antiviral agent, an anti-HIV agent, a non-nucleic acid-based reverse transcriptase inhibitor, an anticancer agent for external use and/or a disinfectant, the dose of steroidal anti-inflammatory agents can be suppressed. Therefore, there are no adverse side effects of steroidal anti-inflammatory agents upon the treatment of oral cavity diseases such as periodontitis, gingivitis, periodontal diseases and stomatitis, and the treatment of skin diseases such as atopic, bacterial, viral and contact diseases, reddening, erythema, ulcer, blister, erosion, trauma and autoimmune diseases. According to the third to fifth embodiments of the present invention, since the composition can sufficiently exhibit anti-inflammatory effects and the like without having the adverse side effects of steroidal anti-inflammatory agents, the composition is particularly useful for the treatment of the oral cavity diseases such as periodontitis and the treatment of atopic skin diseases and the like as mentioned above.

Furthermore, according to the first and third to sixth embodiments of the present invention, a soft, highly water-absorbent polymer has an action of removing the dental plaque attached to the teeth by surface-activating, instead of the abrasive included in the dentrifice. Therefore, particularly when the composition is used as a composition for oral cavity, the softness can prevent wear of the teeth, and therefore the polymer enhances the cleaning effect without peeling off enamel of the teeth, so that the occurrence of dental caries due to a brushing operation can be prevented. Furthermore, when the highly water-absorbent polymer or cellulose derivative that has been conventionally used as a gelling agent acts as a water-absorbent at the affected area, body fluid of the inflamed area can be discharged out, and thus the synergistic action of the medicinal component and the highly water-absorbent polymer or cellulose derivative can further accelerate healing of the affected area.

According to the seventh embodiment of the present invention, since calcium ions act as a conditioning agent that makes the oral cavity alkaline, dissolution of enamel can be avoided, and thus dental caries can be prevented.

According to the eighth embodiment of the present invention, the antihistamine agent suppresses the action of histamine and therefore has an effect of alleviating allergic symptoms in addition to the antibacterial, anti-inflammatory effects and the like. Thus, the composition can suppress atopic dermatitis or allergic diseases such as ulticaria.

According to the ninth embodiment of the present invention, since the composition further contains an antiphlogistic agent, the amount of use of steroidal anti-inflammatory agents that have adverse side effects can be further reduced.

### BRIEF DESCRIPTION OF DRAWINGS

FIG. 1 is a schematic explanatory diagram showing the use of the composition according to the present exemplary embodiment at an inflamed area.

### DESCRIPTION OF REFERENCE NUMERALS

1 INFLAMED AREA
2 HIGHLY WATER-ABSORBENT POLYMER
3 ANTIBACTERIAL AGENT

### BEST MODE FOR CARRYING OUT THE INVENTION

Hereinafter, exemplary embodiments of the present invention will be explained.

Examples of the medicament used in the present invention include an antibacterial agent, an antiviral agent, an anti-HIV agent, a non-nucleic acid-based reverse transcriptase inhibitor, an anticancer agent for external use, and a disinfectant. However, among these, the antibacterial agent, particularly a new quinolone-based antibacterial agent, inhibits the activity of a DNA gyrase, or the like, and thereby acts in a bactericidal manner by interrupting DNA replication of bacteria. Furthermore, since a new quinolone-based antibacterial agent has a wide antibacterial spectrum and a potent antibacterial activity while having almost no adverse side effects, the antibacterial agent can be adapted to various infections. Examples of the new quinolone-based antibacterial agent used in the exemplary embodiment of the present invention include levofloxacin, tosufloxacin, gatifloxacin, sparfloxacin, ciprofloxacin, lomefloxacin, ofloxacin, cinoxacin, norfloxacin, fleroxacin, grepafloxacin, prulifloxacin, sitafloxacin, pazufloxacin, gemifloxacin, moxifloxacin, olamufloxacin and the like, and preferred examples include levofloxacin, tosufloxacin, gatifloxacin, sparfloxacin and ciprofloxacin. The amount of incorporation of the new quinolone-based antibacterial agent is 0.01 to 4.5% by mass, and preferably 1.0 to 2.2% by mass, based on the total amount of the composition.

The new quinolone-based antibacterial agent according to the exemplary embodiment of the present invention may be incorporated singly, or may be incorporated in combination of two or more kinds.

In the exemplar embodiment of the present invention, an antibiotic substance may be used in substitution of the antibacterial agent or in addition to the antibacterial agent, and examples thereof include penicillin family drugs such as penicillin, meticillin, amoxicillin, ampicillin and cloxacillin; cefam family drugs such as cefazolin, cefotiam, ceftriaxone and ceftazidime; aminoglycoside family drugs such as kanamycin, streptomycin, neomycin and gentamycin; tetracycline family drugs such as tetracycline and doxycycline; macrolide family drugs; chloramphenicol family drugs; and the like. Furthermore, in the exemplary embodiment of the present invention, a disinfectant may be used in substitution of the antibacterial agent or in addition to the antibacterial agent, and examples thereof include iodine, sodium hypochlorite, triclosacsan, benzalkonium chloride, benzethonium chloride, acrinol, chlorhexidine, and the like.

A non-steroidal anti-inflammatory agent inhibits the synthesis of prostaglandin, which has inflammatory and pyretic action, and thus has an anti-inflammatory action. Examples of the non-steroidal anti-inflammatory agent used in the exemplary embodiment of the present invention include compounds of indoleacetic acid family (indomethacin and the like), phenylacetic acid family (diclofenac, fenbufen and the like), salicylic acid family (sodium salicylate, methyl salicylate, aspirin and the like), propionic acid family (ibuprofein, flurbiprofen, ketoprofen and the like) and anthranilic acid family (flufenamic acid, mefenamic acid, acetaminophen), but indomethacin, which is an indoleacetic acid-based compound, and diclofenac, which is a phenylacetic acid-based compound, are preferred. The amount of incorporation of the non-steroidal anti-inflammatory agent is preferably 0.01 to 4.5% by mass, and more preferably 0.5 to 2.0% by mass, based on the total amount of the composition.

The non-steroidal anti-inflammatory agent according to the exemplary embodiment of the present invention may be incorporated singly, or may be incorporated in combination of two or more kinds.

A steroidal anti-inflammatory agent suppresses the function of lymphocytes, and thus has an action of strongly suppressing inflammation. Examples of the steroidal anti-inflammatory agent used in the exemplary embodiment of the present invention include dexamethasone, betamethasone, prednisolone, methylprednisolone, hydrocortisone, flumethasone, beclomethasone, momethasone, ciclesonide, dexamethasone, and the like, but preferred examples include dexamethasone and betamethasone. The amount of incorporation of the steroidal anti-inflammatory agent is 0.001 to 4.5% by mass, and preferably 0.01 to 1.0% by mass, based on the total amount of the composition.

The steroidal anti-inflammatory agent according to the exemplary embodiment of the present invention may be incorporated singly, or may be incorporated in combination of two or more kinds.

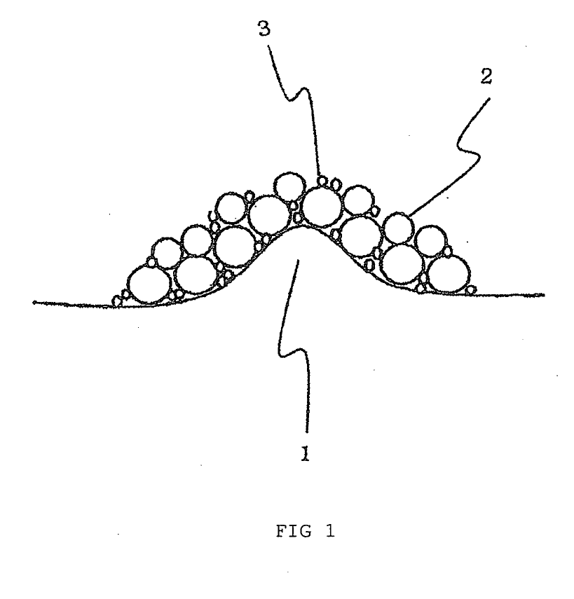

In the exemplary embodiment of the present invention, a highly water-absorbent polymer is not incorporated merely as a gelling agent as in the conventional practice, but is incorporated in substitution of an abrasive that removes dental plaque. Unlike the conventional abrasives (for example, silicic acid, alumina, calcium phosphate, calcium carbonate and the like), the highly water-absorbent polymer peels dental plaque off the teeth in a manner of wrapping the plaque. Thus, the highly water-absorbent polymer does not induce removal of the enamel of the teeth and exposure of the dentine, and is appropriate for the prevention of the cause of pain or dental caries. Furthermore, as shown in FIG. 1, a composition containing a highly water-absorbent polymer, an antibacterial agent, a non-steroidal anti-inflammatory agent and a steroidal anti-inflammatory agent, dries the part applied with the composition as a result of its water-absorbent action so that a penetration pressure is exerted, and thereby body fluid of the inflamed area can be discharged out. Thus, this composition can accelerate healing of the affected area through a synergistic action of the highly water-absorbent polymer or a cellulose derivative and the medicinal component.

Examples of the highly water-absorbent polymer used in the exemplary embodiment of the present invention include vinylic polymers such as carboxyvinyl polymers, polyvinyl alcohol, poly-N-vinylacetamide and polyvinyl methyl ether; acrylic polymers such as polyacrylate, acrylic acid/alkylacrylic copolymers and polyacrylamide; and in addition thereto, starch-acrylic acid graft, isobutylene-maleic acid and the like. In particular, carboxyvinyl polymers, polyvinyl alcohol and polyacrylate are preferred. The amount of incorporation of the highly water-absorbent polymer is 0.001 to 20% by mass, preferably 0.001 to 10% by mass, and more preferably 5.0 to 10% by mass, based on the total amount of the composition. As another embodiment, polymer compounds exemplified by cellulose derivatives such as carboxymethylcellulose, hydroxymethylcellulose, methylcellulose, ethylcellulose and hydroxypropylcellulose; proteins such as gelatin, casein and collagen; and polysaccharides such as bean gum, may be used as water-absorbents or gelling agents in place of the highly water-absorbent polymer. The amount of incorporation of the polymer compound is 0.001 to 20% by mass, preferably 0.001 to 10% by mass, and more preferably 5.0 to 10% by mass, based on the total amount of the composition.

The highly water-absorbent polymer according to the exemplary embodiment of the present invention may be incorporated singly, or may be incorporated in combination of two or more kinds.

In the exemplary embodiment of the present invention, calcium ions may be used as an adjusting agent which makes the inside of the oral cavity alkaline, such as greater than pH 7. It is preferable that the calcium ions be contained in the composition in the form of calcium oxide or calcium hydroxide, but the substance is not particularly limited as long as it is likely to release calcium ions. Furthermore, calcium ions also have an effect of attempting bone recalcification of periodontitis, in addition to rendering the oral cavity alkaline.

The amount of incorporation of calcium ions used in the exemplary embodiment of the present invention is preferably 0.001 to 1.0% by mass, and more preferably 0.01 to 1.0% by mass, based on the total amount of the composition, in order to adjust the pH inside oral cavity to greater than 7.

In the exemplary embodiment of the present invention, an antihistamine agent can be further incorporated to obtain an antibacterial action, an anti-inflammatory action and the like, as well as to suppress allergic diseases. Examples of the antihistamine agent used in the exemplary embodiment of the present invention include chlorphenylamine maleate, diphenhydrazine hydrochloride, mequitazine, promethazine, diphenylpyraline hydrochloride, clemastin fumarate, and the like. The amount of incorporation of the antihistamine agent is 0.001 to 20% by mass, preferably 0.05 to 5.0% by mass, and more preferably 0.01 to 0.1% by mass, based on the total amount of the composition. The antihistamine agent may be incorporated only singly, or may be incorporated in combination of two or more kinds. Furthermore, if necessary, an antiphlogistic agent such as tacrolimus hydrate, ethyl aminobenzoate, bismuth subgallate, camphor, zinc chloride or crotamiton may be incorporated in an amount of 0.01 to 20% by mass based on the total amount of the composition.

As the dosage forms applicable when the composition of the exemplary embodiment of the present invention is used for the oral cavity, a toothpaste, an ointment, a cream, a paste, a direct application using a syringe, a mouthwash, a chewing gum, a troche and the like can be applied. As the dosage forms applicable when the composition is used for the skin, in the case of preparing a preparation for external use, an ointment, a cream, a paste, a patch and the like can be applied, and in the case of preparing an oral medicine, tablets, granules, capsules, a syrup and the like can be applied.

In the case of a toothpaste composition, for example, one or two or more among sorbitol, glycerin, propylene glycol and the like can be incorporated as wetting agents, and one or two or more of sodium alginate, methylcellulose, carboxymethylcellulose, hydroxyethylcellulose and the like can be incorporated as binding agents.

When the composition according to the exemplary embodiment of the present invention is used as a gel, examples of the base include the highly water-absorbent polymer mentioned above, such as a carboxyvinyl polymer, or the gelling agent mentioned above, such as methylcellulose. When the composition is used as an ointment, the base is a hydrophobic one, and examples thereof include petrolatum, paraffin, Plastibase produced by gelling a polyethylene resin, beeswax, spermaceti and the like. In the case of an ointment for oral cavity, the ointment needs to be well attachable to the buccal mucosa and become hardly flowable with saliva or the like, and thus it is preferable to increase adhesiveness by adding celluloses to a hydrophobic base. In the case of using the composition as a cream, esters such as polyethylene glycol are used as emulsifiers. Furthermore, the ointment and cream may be added as necessary with antiseptics such as paraoxybenzoic acid esters, O-phenylphenol and dehydroacetic acid; antioxidants such as dibutylhydroxytoluene, tocopherol and ascorbic acid; chelating agents such as EDTA; and fragrances such as mentha oil, cinnamon oil, eucalyptus oil, spearmint oil, peppermint oil and menthol.

In addition to those, components such as an ultraviolet absorbent, a colorant and a percutaneous absorption accelerating agent can be used as necessary in the ointment and cream. Of course, the components that can be used in the ointment and cream can be appropriately used in skin preparations for external use in general.

In the exemplary embodiment of the present invention, further examples of the medicinal components for toothpaste which may be incorporated, include fluorine compounds having an dental caries preventing effect, such as sodium fluoride, potassium fluoride, ammonium fluoride, tin fluoride and sodium monofluorate; hydroxyapatite having a dental plaque eliminating effect; dental plaque degrading enzymes such as and dextranase; and the like. Also, a vitamin preparation or the like may be further incorporated as an active ingredient for the ointment and cream.

The composition for oral cavity and skin of the exemplary embodiment of the present invention can have a surfactant, a sweetener and other components further incorporated therein, and the composition can be produced by a conventional method, using appropriate components that are conventionally used.

In the exemplary embodiment described above, the composition for oral cavity and skin is made to contain an antibacterial agent and a disinfectant, but medicaments are definitely not limited to these, and a composition for oral cavity and skin may also be prepared by incorporating, in accordance with symptoms, a medicament such as an antiviral agent, an anti-HIV agent, a non-nucleic acid-based reverse transcriptase inhibitor, or an anticancer agent for external use, into the non-steroidal anti-inflammatory agent, steroidal anti-inflammatory agent and highly water-absorbent polymer as described above. As the medicament, those conventionally used can be incorporated.

### Example 1

Next, the present invention will be specifically explained by presenting Experimental Examples. However, the scope of the present invention is not intended to be limited to these.

In Example 1, gels for oral cavity of Invented Examples were prepared by a conventional method according to the prescriptions shown in Table 1 and Table 2, and the gels were evaluated for periodontitis, stomatitis and adverse side effects by the evaluation method described below. In addition, gels for oral cavity of Comparative Examples were prepared by a conventional method according to the prescriptions show in Table 3 and Table 4, and the gels were subjected to the same evaluation. The amount of incorporation is expressed in % by mass.

TABLE 1

| Invented Example | 1 | 2 | 3 | 4 | 5 | 6 | 7 | 8 | 9 | 10 |
|---|---|---|---|---|---|---|---|---|---|---|
| Levofloxacin | 4.1 | 3.0 | 2.0 | — | — | — | — | — | — | — |
| Tosufloxacin | — | — | — | 1.8 | 1.0 | 0.6 | — | — | — | — |
| Gatifloxacin | — | — | — | — | — | — | 1.5 | 1.1 | 0.5 | — |
| Sparfloxacin | — | — | — | — | — | — | — | — | — | 3.8 |
| Ciprofloxacin | — | — | — | — | — | — | — | — | — | — |
| Metronidazole | — | — | — | — | — | — | — | — | — | — |
| Ciprofloxacin | — | — | — | — | — | — | — | — | — | — |
| Chlorhexidine | — | — | — | — | — | — | — | — | — | — |
| Indomethacin | 3.4 | 2.3 | 2.1 | — | — | — | — | — | — | 0.4 |
| Diclofenac | — | — | — | 0.8 | 0.9 | 0.4 | 1.4 | 1.7 | 1.1 | — |
| Dexamethasone | 4.1 | — | — | 0.6 | — | — | 0.08 | — | — | 0.3 |
| Betamethasone | — | 3.0 | — | — | 0.4 | — | — | 0.07 | — | — |
| Prednisone | — | — | 1.8 | — | — | 0.3 | — | — | 0.09 | — |
| Polyacrylic acid | 7.0 | 7.0 | 7.0 | 7.0 | 7.0 | 7.0 | 7.0 | 7.0 | 7.0 | 7.0 |
| Calcium hydroxide | 0.1 | 0.1 | 0.1 | 0.1 | 0.1 | 0.1 | 0.1 | 0.1 | 0.1 | 0.1 |
| Methylcellulose | 2.0 | 2.0 | 2.0 | 2.0 | 2.0 | 2.0 | 2.0 | 2.0 | 2.0 | 2.0 |
| Purified water | Balance | Balance | Balance | Balance | Balance | Balance | Balance | Balance | Balance | Balance |
| Total | 100.0 | 100.0 | 100.0 | 100.0 | 100.0 | 100.0 | 100.0 | 100.0 | 100.0 | 100.0 |

| Invented Example | 11 | 12 | 13 | 14 | 15 | 16 | 17 | 18 |
|---|---|---|---|---|---|---|---|---|
| Levofloxacin | — | — | — | — | — | — | — | — |
| Tosufloxacin | — | — | — | — | — | — | — | — |
| Gatifloxacin | — | — | — | — | — | — | — | — |
| Sparfloxacin | 2.8 | 2.2 | — | — | — | — | — | — |
| Ciprofloxacin | — | — | 4.1 | 3.4 | 2.3 | — | — | — |
| Metronidazole | — | — | 2.0 | 2.0 | 2.0 | — | — | — |
| Ciprofloxacin | — | — | — | — | — | 1.7 | 1.0 | 0.4 |
| Chlorhexidine | — | — | — | — | — | 0.05 | 0.05 | 0.05 |
| Indomethacin | 0.2 | 0.3 | 0.7 | 0.7 | 0.6 | — | — | — |
| Diclofenac | — | — | — | — | — | 0.2 | 0.4 | 0.3 |
| Dexamethasone | — | — | 0.3 | — | — | 0.09 | — | — |
| Betamethasone | 0.2 | — | — | 0.3 | — | — | 0.07 | — |
| Prednisone | — | 0.2 | — | — | 0.3 | — | — | 0.02 |
| Polyacrylie acid | 7.0 | 7.0 | 7.0 | 7.0 | 7.0 | 7.0 | 7.0 | 7.0 |
| Calcium hydroxide | 0.1 | 0.1 | 0.1 | 0.1 | 0.1 | 0.1 | 0.1 | 0.1 |
| Methylcellulose | 2.0 | 2.0 | 2.0 | 2.0 | 2.0 | 2.0 | 2.0 | 2.0 |
| Purified water | Balance | Balance | Balance | Balance | Balance | Balance | Balance | Balance |
| Total | 100.0 | 100.0 | 100.0 | 100.0 | 100.0 | 100.0 | 100.0 | 100.0 |

TABLE 2

| Invented Example | 19 | 20 | 21 | 22 | 23 | 24 | 25 | 26 | 27 | 28 | 29 | 30 |
|---|---|---|---|---|---|---|---|---|---|---|---|---|
| Benzalkonium | 0.1 | 0.07 | 0.06 | — | — | — | — | — | — | — | — | — |
| chloride | | | | | | | | | | | | |
| Sodium | — | — | — | 0.02 | 0.15 | 0.07 | — | — | — | — | — | — |
| hypochlorite | | | | | | | | | | | | |
| Chlorhexidine | — | — | — | — | — | — | 0.02 | 0.15 | 0.07 | — | — | — |
| gluconate | | | | | | | | | | | | |
| Iodine | — | — | — | — | — | — | — | — | — | 0.1 | 0.1 | 0.1 |
| Indomethacin | 3.4 | 2.3 | 2.1 | — | — | — | — | — | — | 0.4 | 0.2 | 0.3 |
| Diclofenac | — | — | — | 0.8 | 0.9 | 0.4 | 1.4 | 1.7 | 1.1 | — | — | — |
| Dexamethasone | 4.1 | — | — | 0.6 | — | — | 0.08 | — | — | 0.3 | — | — |
| Betamethasone | — | 3.0 | — | — | 0.4 | — | — | 0.07 | — | — | 0.2 | — |
| Prednisone | — | — | 1.8 | — | — | 0.3 | — | — | 0.09 | — | — | 0.2 |
| Polyacrylic acid | 7.0 | 7.0 | 7.0 | 7.0 | 7.0 | 7.0 | 7.0 | 7.0 | 7.0 | 7.0 | 7.0 | 7.0 |
| Clacium hydroxide | 0.1 | 0.1 | 0.1 | 0.1 | 0.1 | 0.1 | 0.1 | 0.1 | 0.1 | 0.1 | 0.1 | 0.1 |
| Methylcellulose | 2.0 | 2.0 | 2.0 | 2.0 | 2.0 | 2.0 | 2.0 | 2.0 | 2.0 | 2.0 | 2.0 | 2.0 |
| Purified water | Balance | Balance | Balance | Balance | Balance | Balance | Balance | Balance | Balance | Balance | Balance | Balance |
| Total | 100.0 | 100.0 | 100.0 | 100.0 | 100.0 | 100.0 | 100.0 | 100.0 | 100.0 | 100.0 | 100.0 | 100.0 |

TABLE 3

| Comparative Example | 1 | 2 | 3 | 4 |
|---|---|---|---|---|
| Levofloxacin | 0.005 | — | 2.0 | — |
| Tosufloxacin | — | 0.005 | — | 2.0 |
| Indomethacin | 0.005 | — | 0.6 | — |
| Diclofenac | — | 0.005 | — | 0.6 |
| Dexamethasone | 5.0 | — | 5.0 | — |
| Betamethasone | — | 6.0 | — | 6.0 |
| Polyacrylic acid | 7.0 | 7.0 | 7.0 | 7.0 |
| Calcium hydroxide | 0.1 | 0.1 | 0.1 | 0.1 |
| Methylcellulose | 2.0 | 2.0 | 2.0 | 2.0 |
| Purified water | Balance | Balance | Balance | Balance |
| Total | 100.0 | 100.0 | 100.0 | 100.0 |

TABLE 4

| Comparative Example | 5 | 6 | 7 | 8 |
|---|---|---|---|---|
| Calcium hypochlorite | 0.005 | — | 0.5 | — |
| Sodium hypochlorite | — | 0.005 | — | 0.5 |
| Indomethacin | 0.005 | — | 0.6 | — |
| Diclofenac | — | 0.005 | — | 0.6 |
| Dexamethasone | 5.0 | — | 5.0 | — |
| Betamethasone | — | 6.0 | — | 6.0 |
| Polyacrylic acid | 7.0 | 7.0 | 7.0 | 7.0 |
| Calcium hydroxide | 0.1 | 0.1 | 0.1 | 0.1 |
| Methylcellulose | 2.0 | 2.0 | 2.0 | 2.0 |
| Purified water | Balance | Balance | Balance | Balance |
| Total | 100.0 | 100.0 | 100.0 | 100.0 |

Evaluation Method

For each of the Blend Examples, 10 male testees and 10 female testees, 20 people in total, were instructed to brush the teeth after every meal, subsequently to wipe the affected area and the surroundings with cotton or the like, and to apply 1 g of the composition on the affected area with a finger. This was continued for one week. The experimental results were obtained by observing the affected area and determining the number of testees under the following classifications. The numbers of people are presented in Table 5 and Table 6 for the Invented Examples and in Table 7 and Table 8 for the Comparative Examples. The presence or absence of adverse side effects was determined on the basis of the presence or absence of any abnormalities in the affected area, such as pain in the site of administration or mucosal disorder.

Effective in periodontitis and stomatitis: ◯

Slightly effective in periodontitis and stomatitis: Δ

Not effective in periodontitis and stomatitis: x

Adverse side effects present: Y

Adverse side effects absent: N

TABLE 5

| | Invented Example | | | | | | | | | | | | | | | | | |
|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 |
| ◯ | 18 | 15 | 16 | 12 | 13 | 12 | 17 | 16 | 15 | 17 | 15 | 16 | 17 | 17 | 17 | 13 | 14 | 13 |
| Δ | 2 | 3 | 3 | 6 | 5 | 6 | 2 | 3 | 4 | 2 | 4 | 3 | 3 | 2 | 3 | 4 | 3 | 4 |
| X | 0 | 2 | 1 | 2 | 2 | 2 | 1 | 1 | 1 | 1 | 1 | 1 | 0 | 1 | 0 | 3 | 3 | 3 |
| Total | 20 | 20 | 20 | 20 | 20 | 20 | 20 | 20 | 20 | 20 | 20 | 20 | 20 | 20 | 20 | 20 | 20 | 20 |
| Y | 0 | 0 | 0 | 0 | 0 | 0 | 0 | 0 | 0 | 0 | 0 | 0 | 0 | 0 | 0 | 0 | 0 | 0 |
| N | 20 | 20 | 20 | 20 | 20 | 20 | 20 | 20 | 20 | 20 | 20 | 20 | 20 | 20 | 20 | 20 | 20 | 20 |
| Total | 20 | 20 | 20 | 20 | 20 | 20 | 20 | 20 | 20 | 20 | 20 | 20 | 20 | 20 | 20 | 20 | 20 | 20 |

TABLE 6

| | Invented Example | | | | | | | | | | | |
|---|---|---|---|---|---|---|---|---|---|---|---|---|
| | 19 | 20 | 21 | 22 | 23 | 24 | 25 | 26 | 27 | 28 | 29 | 30 |
| ◯ | 17 | 15 | 16 | 13 | 14 | 12 | 16 | 16 | 16 | 17 | 16 | 16 |
| Δ | 3 | 3 | 2 | 5 | 5 | 6 | 3 | 2 | 3 | 2 | 3 | 3 |
| X | 0 | 2 | 2 | 2 | 1 | 2 | 1 | 2 | 1 | 1 | 1 | 1 |
| Total | 20 | 20 | 20 | 20 | 20 | 20 | 20 | 20 | 20 | 20 | 20 | 20 |
| Y | 0 | 0 | 0 | 0 | 0 | 0 | 0 | 0 | 0 | 0 | 0 | 0 |
| N | 20 | 20 | 20 | 20 | 20 | 20 | 20 | 20 | 20 | 20 | 20 | 20 |
| Total | 20 | 20 | 20 | 20 | 20 | 20 | 20 | 20 | 20 | 20 | 20 | 20 |

TABLE 7

| Comparative Example | 1 | 2 | 3 | 4 |
|---|---|---|---|---|
| ◯ | 4 | 5 | 16 | 17 |
| Δ | 7 | 4 | 2 | 1 |
| X | 9 | 11 | 2 | 2 |
| Total | 20 | 20 | 20 | 20 |
| Y | 6 | 8 | 7 | 8 |
| N | 14 | 12 | 13 | 12 |
| Total | 20 | 20 | 20 | 20 |

TABLE 8

| Comparative Example | 5 | 6 | 7 | 8 |
|---|---|---|---|---|
| ◯ | 4 | 5 | 16 | 17 |
| Δ | 5 | 5 | 3 | 1 |
| X | 11 | 10 | 1 | 2 |
| Total | 20 | 20 | 20 | 20 |
| Y | 5 | 8 | 6 | 8 |
| N | 15 | 12 | 14 | 12 |
| Total | 20 | 20 | 20 | 20 |

As it can be seen from these results, compositions containing 0.01 to 4.5% by mass of an antibacterial or a disinfectant, 0.01 to 4.5% by mass of a non-steroidal anti-inflammatory agent, and 0.001 to 4.5% by mass of a steroidal anti-inflammatory agent, does not undergo a reduction in the anti-inflammatory action, and thus can heal periodontitis while avoiding adverse side effects.

### Example 2

In Example 2, anti-dermatitis gels and antifungal gels of Invented Examples were prepared by conventional methods according to the prescriptions shown in Table 1 and Table 2 shown above, and the gels were evaluated for dermatitis, ringworm and adverse side effects by the evaluation method described below. In addition, anti-dermatitis gels and antifungal gels of Comparative Examples were prepared by conventional methods according to the prescriptions show in Table 3 and Table 4 shown above, and the gels were subjected to the same evaluations. The amount of incorporation is expressed in % by mass.

Evaluation Method

For each of the Blend Examples, 5 male testees and 5 female testees, 10 people in total, were instructed to clean the affected area and the surroundings with cotton or the like, and to apply 1 g of the composition on the affected area with a finger. This was continued for one week. The experimental results were obtained by observing the affected area and determining the number of testees under the following classifications. The numbers of people are presented in Table 9 and Table 10 for the Invented Examples and in Table 11 and Table 12 for the Comparative Examples. The presence or absence of adverse side effects was determined on the basis of the presence or absence of any abnormalities in the affected area, such as pain in the site of administration.

Effective in dermatitis and ringworm: ◯

Not effective in dermatitis and ringworm: x

Adverse side effects present: Y

Adverse side effects absent: N

TABLE 9

| | Invented Example | | | | | | | | | | | |
|---|---|---|---|---|---|---|---|---|---|---|---|---|
| | 19 | 20 | 21 | 22 | 23 | 24 | 25 | 26 | 27 | 28 | 29 | 30 |
| ◯ | 10 | 10 | 10 | 10 | 10 | 10 | 10 | 10 | 9 | 10 | 9 | 10 |
| X | 0 | 0 | 0 | 0 | 0 | 0 | 0 | 0 | 1 | 0 | 1 | 0 |
| Total | 10 | 10 | 10 | 10 | 10 | 10 | 10 | 10 | 10 | 10 | 10 | 10 |
| Y | 0 | 0 | 0 | 0 | 0 | 0 | 0 | 0 | 0 | 0 | 0 | 0 |
| N | 10 | 10 | 10 | 10 | 10 | 10 | 10 | 10 | 10 | 10 | 10 | 10 |
| Total | 10 | 10 | 10 | 10 | 10 | 10 | 10 | 10 | 10 | 10 | 10 | 10 |

TABLE 10

| | Invented Example | | | | | | | | | | | |
|---|---|---|---|---|---|---|---|---|---|---|---|---|
| | 19 | 20 | 21 | 22 | 23 | 24 | 25 | 26 | 27 | 28 | 29 | 30 |
| ◯ | 10 | 10 | 10 | 10 | 10 | 10 | 10 | 10 | 9 | 10 | 9 | 10 |
| X | 0 | 0 | 0 | 0 | 0 | 0 | 0 | 0 | 1 | 0 | 1 | 0 |
| Total | 10 | 10 | 10 | 10 | 10 | 10 | 10 | 10 | 10 | 10 | 10 | 10 |
| Y | 0 | 0 | 0 | 0 | 0 | 0 | 0 | 0 | 0 | 0 | 0 | 0 |
| N | 10 | 10 | 10 | 10 | 10 | 10 | 10 | 10 | 10 | 10 | 10 | 10 |
| Total | 10 | 10 | 10 | 10 | 10 | 10 | 10 | 10 | 10 | 10 | 10 | 10 |

TABLE 11

| Comparative Example | 1 | 2 | 3 | 4 |
|---|---|---|---|---|
| ◯ | 5 | 4 | 9 | 10 |
| X | 5 | 6 | 1 | 0 |
| Total | 10 | 10 | 10 | 10 |
| Y | 3 | 5 | 3 | 6 |
| N | 7 | 5 | 7 | 4 |
| Total | 10 | 10 | 10 | 10 |

TABLE 12

| Comparative Example | 5 | 6 | 7 | 8 |
|---|---|---|---|---|
| ◯ | 4 | 4 | 10 | 10 |
| X | 6 | 6 | 1 | 0 |
| Total | 10 | 10 | 10 | 10 |
| Y | 3 | 4 | 4 | 6 |
| N | 7 | 6 | 6 | 4 |
| Total | 10 | 10 | 10 | 10 |

From these results, dermatitis and ringworm were also healed with compositions containing 0.01 to 4.5% by mass of an antibacterial agent or a disinfectant, as well as 0.01 to 4.5% by mass of a non-steroidal anti-inflammatory agent and 0.001 to 4.5% by mass of a steroidal anti-inflammatory agent, and adverse side effects could also be avoided.

### INDUSTRIAL APPLICABILITY

The composition of the present invention can maintain an anti-inflammatory action while reducing adverse side effects, and also can be incorporated into not only toothpaste but also ointment and the like. Thus, the composition can be used as a composition for oral cavity against periodontitis and stomatitis, as well as a composition for skin against allergic dermatitis.

1. A composition for oral cavity and skin, comprising 0.01 to 4.5% by mass of at least one selected from the group consisting of an antibacterial agent, an antiviral agent, an anti-HIV agent, a non-nucleic acid-based reverse transcriptase inhibitor, an anticancer agent for external use and a disinfectant; 0.01 to 4.5% by mass of a non-steroidal anti-inflammatory agent; 0.001 to 4.5% by mass of a steroidal anti-inflammatory agent; and 0.001 to 10% by mass of a highly water-absorbent polymer or a cellulose derivative.

2. The composition for oral cavity and skin according to claim 1, comprising, in substitution of the absorbent polymer, 0.001 to 10% by mass of a gelling agent, and 0.001 to 1.0% by mass of calcium hydroxide, calcium oxide or a calcium compound that is prone to release calcium ions.

3. The composition for oral cavity and skin according to claim 1, wherein the above antibacterial agent was chosen among aspoxicillin, amoxicillin, ampicillin, ciclacillin, talampicillin, sultamicillin tosilate, bacampicillin, pivmecillinam, piperacillin sodium, phenethicillin potassium, benzylpenicillin potassium, benzyl-penicillin benzathine, lenampicillin, cefaclor, cefazolin sodium, cefatrizine propylene glycol, cefadroxil, cefalexin, cefalothin sodium, cefixime, cefepime, cefodizime sodium, cefotaxime sodium, cefotiam, cefotiam hexetil, cefotetan, cefozopran, cefoperazone sodium, cefcapene pivoxil, cefditoren pivoxil, cefdinir, cefsulodin sodium, ceftazidime, ceftizoxime sodium, ceftibuten, cefteram pivoxil, ceftriaxone sodium, cefpiramide sodium, cefpirome, cefbuperazone sodium, cefpodoxime proxetil, cefminox sodium, cefmetazole sodium, cefmenoxime, cefroxadine, cefuroxime axetil, flomoxef sodium, latamoxef sodium, aztreonam, carumonam sodium, imipenem.cilastatin sodium, panipenem.betamipron, biapenem, meropenem, doripenem, faropenem sodium, astromicin, amikacin, arbekacin, isepamicin, kanamycin, gentamicin, erythromycin, sisomicin, dibekacin, streptomycin, spectinomycin, vancomycin, nalidixic acid, nafcillin, oxacillin, levofloxacin, tosufloxacin, gatifloxacin, sparfloxacin, Garenoxacin, enoxacin, ciprofloxacin, ofloxacin, cinoxacin, sulfamethoxazole.trimethoprim, norfloxacin, pazufloxacin, pipemidic acid, pipemidic acid trihydrate, fleroxacin, prulifloxacin, linezolid, lomefloxacin, telithromycin, sulfadimethoxine, sulfamonomethoxine, alumino-p-aminosalicylate, isoniazid, ethionamide, ethambutol, enviomycin, cycloserine, calcium para-aminosalicylate, pyrazinamide, rifampicin, clofazimine, diaphenylsulfone, pentamidine isetionate, amphotericin B, nystatin, flucytosine, miconazole, itraconazole, fluconazole, fosfluconazole, voriconazole, terbinafine, micafungin sodium, griseofulvin, metronidazole and tinidazole group; is at least one,
wherein the above antiviral agent is aciclovir, valaciclovir, vidarabine, ganciclovir, valganciclovir hydrochloride, foscarnet sodium hydrate and palivizumab group; is at least one, and above antiHIV agent was chosen among the zidovudine, didanosine, zalcitabine, sanilvudine, lamivudine, abacavir, tenofovir disoproxil fumarate, disoproxil and emtricitabine group at least one, and above non-nucleoside reverse transcriptase inhibitor is efavirenz, nevirapine, protease inhibitor or saquinavir mesilate, saquinavir, ritonavir, praziquantel and sodium antimonyl tartrate group that it is at least one, and above anticancer agent was chosen among fluorouracil, tegafur, bleomycin and the BCG (bacillus calmette and Guerin) group that it is at least one, and an above sterilizer was chosen among Acrinol, the fourth grade ammonium salt, cetylpyridinium chloride, benzalkonium chloride, sodium hypochlorite, calcium hypochlorite, chlorhexidine, Irgasan, phenol and the iodine group that it is at least one, a constituent to claim 1 for oral cavity use and skin of mention.

4. The composition for oral cavity and skin according to claim 1, wherein the non-steroidal anti-inflammatory agent is at least one selected from the group consisting of indomethacin, diclofenac, acemetacin, alclofenac, amfenac, aspirin, bendazac, benorylate, benoxaprofen, bucloxic acid, bufexamac, bumadisone, butibufen, carprofen, cinmetacin, clidanac, clometacin, cloripac, diclofenac, diflunisan, etodolac, etofenamate, felbinac, fenbufen, fenclofenac, fenclorac, fendosal, fenoprofen, fentiazac, flufenamic acid, flurbiprofen, glafenine, ibufenac, ibuprofen, indomethacin, isofezolac, isoxepac, ketoprofen, ketorolac, lonazolac, meclofenamic acid, mefanamic acid, metiazinic acid, nabumetone, naproxen, niflumic acid, oxametacin, oxaprozin, pirazolac, piroxicam, protizinic acid, salicylic acid, sulindac, surgam, tenidap, tenoxicam, tiaramide, tinoridine, tolfenamic acid and tolmetin.

5. The composition for oral cavity and skin according to claim 1, wherein the steroidal anti-inflammatory agent is at least one selected from the group consisting of dexamethasone, betamethasone, cortisone, hydrocortisone, fludrocortisones, methylprednisolone, triamcinolone, paramethasone and prednisone.

6. The composition for oral cavity and skin according to claim 1, wherein the highly water-absorbent polymer is at least one selected from the group consisting of carboxyvinyl polymers, polyvinyl alcohol, polyacrylate, starch-acrylic acid graft, isobutylene-maleic acid and poly-N-vinylacetamide.

7. The composition for oral cavity and skin according to claim 1, which further comprises 0.001 to 1.0% by mass of calcium ions, and has a pH of greater than 7 to 14.

8. The composition for oral cavity and skin according to claim 1, which further comprises 0.001 to 20% by mass of at least one antihistamine agent selected from the group consisting of chlorpheniramine maleate, diphenhydrazine hydrochloride, clemastin fumarate, triprolidine hydrochloride, alimemazine tartrate, promethazine hydrochloride, homochlorcyclizine hydrochloride, hydroxyzine and cyproheptadine hydrochloride.

9. The composition for oral cavity and skin according to claim 1, which further contains 0.01 to 20% by mass of at least one antiphlogistic agent selected from the group consisting of tacrolimus hydrate, ethyl aminobenzoate, bismuth subgallate, camphor, zinc chloride and crotamiton.
